(19) United States
(12) Plant Patent Application Publication
(10) Pub. No.: US 2018/0199485 P1
(43) Pub. Date: Jul. 12, 2018

(54) Poinsettia plant named 'NPCW16249'

(50) ;

(71) Applicant: Klemm+Sohn GmbH & Co. KG, Stuttgart (DE)

(72) Inventors: Nils Klemm, Stuttgart (DE); Guido von Tubeuf, Stuttgart (DE)

(73) Assignee: Klemm+Sohn GmbH & Co. KG, Stuttgart (DE)

(21) Appl. No.: 15/530,396

(22) Filed: Jan. 9, 2017

(51) Int. Cl. A01H 5/02

(52) U.S. Cl. PLT/307

(57) ABSTRACT

A new poinsettia plant particularly distinguished by having red bracts and very good keeping quality is disclosed.

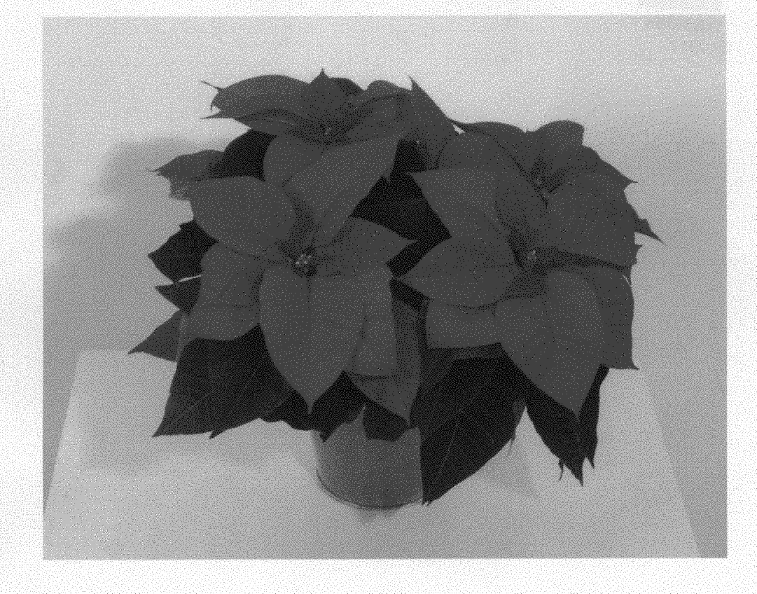

### CROSS-REFERENCE TO RELATED APPLICATIONS

Plant Breeders Rights applications for poinsettia ‘NPCW16249’ were filed in Poland on Feb. 19, 2016 (Application No. O O1880) and in Europe on Apr. 22, 2016 (Application No. 2016/0991). ‘NPCW16249’ has not been made publicly available or sold more than one year prior to the filing date of this application.

Genus and species: Euphorbia pulcherrima.

Variety denomination: ‘NPCW16249’.

### BACKGROUND OF THE NEW PLANT

The present invention comprises a new and distinct variety of poinsettia, botanically known as Euphorbia pulcherrima and hereinafter referred to by the variety name ‘NPCW16249’. ‘NPCW16249’ was derived from a controlled cross conducted in Nairobi, Kenya in November of 2010 between the proprietary and unpatented female variety known as ‘EP-1995-0001’ and the proprietary and unpatented male variety ‘EP-1995-Primero’.

In October 2011, a single plant was selected in Stuttgart, Germany. In April 2013, ‘NPCW16249’ was first asexually propagated by vegetative apical cuttings in Stuttgart Germany. ‘NPCW16249’ was found to reproduce true to type in successive generations of asexual propagation via vegetative apical cuttings in Stuttgart, Germany.

### SUMMARY

The following are the most outstanding and distinguishing characteristics of this new variety when grown under normal horticultural practices in Stuttgart, Germany.
1. Red bracts; and
2. Very good keeping quality.

### DESCRIPTION OF THE PHOTOGRAPH

This new poinsettia plant is illustrated by the accompanying photograph which shows an overall view of the whole plant. The photograph is of a plant approximately 16 to 18 weeks old grown indoors in Stuttgart, Germany in October 2016. The colors shown are as true as can be reasonably obtained by conventional photographic procedures.

### DESCRIPTION OF THE NEW VARIETY

The following detailed descriptions set forth the distinctive characteristics of ‘NPCW16249’. The data which define these characteristics were collected from asexual reproductions carried out in Stuttgart, Germany. Data was collected on plants grown approximately 19 weeks after potting in a greenhouse in Stuttgart, Germany under greenhouse conditions in December 2016. Color references are to The R.H.S. Colour Chart of The Royal Horticultural Society of London (R.H.S.), 5^{th} edition (2007).
Classification:
   Family.—Euphorbiaceae.
   Species.—Euphorbia pulcherrima.
   Common name.—Poinsettia.
   Variety.—‘NPCW16249’.
Parentage:
   Female.—‘EP-1995-0001’ (unpatented).
   Male.—‘EP-1995-Primero’ (unpatented).
Growth:
   Blooming habit.—Intermittent.
   Flowering response time.—Approximately 60 days.
   Keeping quality.—Very good.
Plant description:
   Form.—Triangular.
   Growth habit.—Upright.
   Height.—28.0 cm to 30.0 cm.
   Width.—25.0 cm to 30.0 cm.
   Average number of inflorescences per plant.—6.
Propagation:
   Type cuttings.—Apical cuttings.
   Time to produce a rooted cutting.—4 weeks.
   Rooting habit and root color.—Freely branching and white.
Lateral branches:
   Color.—RHS 143A with a layer of RHS 59A in alternating intensity.
   Length.—25.0 cm to 27.0 cm.
   Internode length.—1.2 cm to 3.5 cm.
   Diameter.—0.5 cm to 0.8 cm.
   Branching habit.—Freely branching.
   Number of lateral branches per plant.—Average of 6.
   Texture.—Smooth.
Leaves:
   Quantity.—6 to 8 per lateral branch.
   Arrangement.—Alternate.
   Number of lobes on the leaf blade.—0 to 2.
   Lobation depth.—Shallow.
   Depth of the deepest sinus of the leaf blade.—0.5 cm.
   Shape.—Ovate.
   Apex.—Acute.
   Base.—Obtuse.
   Margin.—Almost entire.
   Length.—8.0 cm to 13.0 cm.
   Width.—6.0 cm to 9.0 cm.
   Texture (both surfaces).—Smooth.
   Vein color.—Upper surface: Transparent Lower surface: RHS 139D, middle vein slightly reddish.
   Variegation.—Absent.
   Venation pattern.—Reticulate.
   Color.—Mature foliage: Upper surface: RHS 139A Lower surface: RHS 139D Immature foliage: Upper surface: RHS 141A Lower surface: RHS 139D.
   Leaf petiole.—Length: 3.0 cm to 5.5 cm Diameter: 0.2 cm to 0.3 cm Color: Upper (part) surface: RHS 59A Lower (part) surface: RHS 181B Texture: Smooth Strength: Medium.
Inflorescence:
   Lastingness of inflorescence on the plant.—4 weeks.
   Natural flowering season.—Beginning of December.
   Diameter.—15.0 cm to 18.0 cm.
   Height.—Approximately 3.0 cm.
   Fragrance.—Absent.
Bracts:
   Number of bracts per inflorescence.—6 to 8.
   Shape.—Ovate.
   Apex.—Acute.
   Base.—Obtuse.
   Margin.—Entire.
   Size.—Length: 8.0 cm to 10.0 cm Width: 4.0 cm to 8.0 cm.
   Texture (both upper and lower surfaces).—Smooth.
   Venation pattern.—Reticulate.
   Vein color.—Upper surface: RHS 59A Lower surface: RHS 181B.
   Bract color.—Upper surface: RHS 46B Lower surface: RHS 46B.
   Bract petiole.—Length: 0.5 cm to 2.0 cm Diameter: 0.2 cm to 0.3 cm Color: Upper (part) surface: RHS 59A Lower (part) surface: RHS 181B Texture: Smooth Strength: Medium.
Cyme:
   Diameter.—1.0 cm to 2.0 cm.
   Cyathia number.—5 to 8 per inflorescence.
   Deformation of glands of the cyathium.—Absent.
   Timing of opening of cyathia.—Beginning of December. First pollen appears in parallel to opening of the nectar cups; stigmas appear 1 to 2 weeks later.
   Cyathium.—Shape: Ovate Diameter: 0.2 cm to 0.5 cm Length: 0.3 cm to 0.8 cm Color: RHS 143C.
Peduncle: Color: RHS 142B Length: 0.2 cm to 0.3 cm Diameter: 0.1 cm Texture: Smooth Strength: Medium.
   Nectar cups.—Number: 1 per cyathium Diameter: 0.1 cm Size: 0.4 cm Color: RHS 1C.
Reproductive organs:
   Stamen quantity.—5 to 8 per cyathium.
   Stamen shape.—Ovate.
   Filament length.—0.25 cm.
   Filament color.—RHS 46A.
   Pollen quantity.—Sparse.
   Pollen color.—RHS 12A.
   Gynoecium.—Present. Pistil quantity per cyanthium: 1 Pistil length: 0.2 cm Stigma form: Divided in two parts Stigma color: RHS 183A Style length: 0.2 cm Style color: RHS 183A Ovary length: 0.05 cm Ovary color: RHS 143C.
   Fruit and seed set.—Not observed.
   Disease and pest resistance.—No special disease or pest resistance observed.

### COMPARISON WITH PARENTAL LINES AND KNOWN VARIETY

‘NPCW16249’ is a distinct variety of poinsettia having red bracts. ‘NPCW16249’ is distinguished from its parental lines as described in Table 1.

TABLE 1

| | Variety | Female | Male |
|---|---|---|---|
| Characteristic | ‘NPCW16249’ | ‘EP-1995-0001’ | ‘EP-1995-Primero’ |
| Response time | Late season | Midseason | Midseason |
| Vigor | Medium-strong | Compact-medium | Strong |
| Bract color | Red | Orange red | Dark bluish red |

‘NPCW16249’ is distinguished from commercial line ‘Christmas Wish’ (U.S. Plant Pat. No. 26,625) also known as ‘NPCW13218’ as described in Table 2.

TABLE 2

| Characteristic | Variety ‘NPCW16249’ | ‘Christmas Wish’ |
|---|---|---|
| Response time | Late season | Early midseason |
| Bract size | Medium | large |

We claim:

1. A new and distinct variety of poinsettia plant named ‘NPCW16249’ as illustrated and described herein.
